(19) United States
(12) Patent Application Publication
(10) Pub. No.: US 2022/0091093 A1
(43) Pub. Date: Mar. 24, 2022

(54) MXene Nanopore Sequencer of Biopolymers

(71) Applicants: Northeastern University, Boston, MA (US); Auburn University, Auburn, AL (US)

(72) Inventors: Meni WANUNU, Needham, MA (US); Mehrnaz MOJTABAVI, Allston, MA (US); Armin VAHID MOHAMMADI, Auburn, AL (US); Majid BEIDAGHI, Auburn, AL (US)

(21) Appl. No.: 17/424,636

(22) PCT Filed: Feb. 3, 2020
(86) PCT No.: PCT/US2020/016456

§ 371 Date: Jul. 21, 2021

Related U.S. Application Data
(60) Provisional application No. 62/800,390, filed on Feb. 1, 2019.

(51) Int. Cl. G01N 33/487; G01N 27/447

(52) U.S. Cl. CPC G01N 33/48721; G01N 27/44791; G01N 27/4473

(57) ABSTRACT

The present technology provides a nanopore electrode sequencer for the characterization and sequencing of biomolecules. Two or more ultrathin MXene sheets containing nanopores serve as electrodes that bind and store cations which can be released to provide ionic current through the nanopore during sequencing, thereby eliminating access resistance to ions at the entrance to the nanopore from bulk solution. Resolution of ionic current changes caused by biopolymer components within the nanopore is thereby substantially improved, providing more sensitive and robust sequencing of biopolymers.

### CROSS REFERENCE TO RELATED APPLICATIONS

This application claims the priority of U.S. Provisional Application No. 62/800,390 filed 1 Feb. 2019 and entitled “MXene Nanopore Sequencer of Biopolymers”, the whole of which is hereby incorporated by reference.

### STATEMENT REGARDING FEDERALLY SPONSORED RESEARCH OR DEVELOPMENT

This invention was made with government support under Grant No. 1542707 awarded by the National Science Foundation. The government has certain rights in the invention.

### BACKGROUND

In commercially available nanopore technologies for biomolecule sequencing, membrane-embedded proteins are used as sensors. Although these sensors provide precise geometry with high reproducibility and tunability, they typically lack mechanical and chemical robustness, and there is little flexibility with regard to available pore sizes. This limitation can be overcome by replacing such proteins with atomically thin synthetic materials such as two-dimensional materials. Nanopores in two-dimensional materials offer high resolution comparable to proteins for sequencing of biomolecules, along with higher mechanical robustness. However, sequencing with two-dimensional materials is limited in resolution due to access resistance caused by the entrance of ions from bulk solution into the nanopore. Thus, the sensing region is effectively longer than the geometric pore thickness. In order to improve resolution during biomolecule sequencing, there is a need to reduce access resistance at nanopores.

### SUMMARY

The present technology provides a nanopore electrode sequencer for the characterization and sequencing of biomolecules. The technology utilizes two or more MXene sheets or membranes containing nanopores. MXenes are two-dimensional inorganic materials one or more atoms thick and containing transition metal carbides, nitrides, or carbonitrides. The MXene sheets can serve as electrodes that bind and store cations which can be released to provide ionic current through the nanopore during sequencing, thereby reducing or eliminating access resistance to ions at the entrance to the nanopore from bulk solution. Resolution of ionic current changes caused by biopolymer components within the nanopore is thereby substantially improved.

Described herein are two approaches for sequencing of polymers using nanopores in electrically-conducting, ion-intercalating MXene membranes. Both approaches can be used to analyze, including determining the sequence of but also investigating the conformation and function of, any polymer composed of repeating monomeric units, but are especially suited for sequencing single biopolymer molecules or fragments or derivatives thereof.

The first approach is based on ion transport localization between an ultrathin nanopore having intercalated ions (between MXene sheets) and an electrolyte chamber. In this approach access resistance is overcome by using ion-intercalating two-dimensional flakes assembled to form a nanometer-thick membrane and applying voltage to that membrane to release ions directly from within the membrane through the nanopore. In other words, by intercalating the ions between the electrode layers and releasing them by applying reverse voltage, ions can travel to electrolyte chamber without facing any access resistance. This approach is expected to significantly improve the sensing resolution by overcoming the access resistance limitation and can form the foundation for a new type of nanopore-based DNA/RNA/protein sequencing using solid-state nanopores. The process is reversible, and it is possible to recapture ions by re-intercalation.

In the second approach, ion transport localization between two ultrathin ion-intercalating MXene electrodes provides a finite path for ions to afford true single base resolution and overcoming of access resistance. This approach uses a device that includes two electrode layers. Each electrode layer comprises a sandwich of two MXene sheet layers that has alkali ions intercalated in the interstitial region. Between the two electrodes a dielectric gap exists, produced by a known deposition method, e.g., atomic-layer deposition. Application of voltage between the two electrode layers promotes ion transport from one electrode to the other. Since both electrodes consist of ion reservoirs in their interstitial region, ions can traverse the pore without facing any access resistance, thereby allowing achievement of high resolution in biopolymer sequencing. The methods and devices described here also provide scalability solutions for making arrays of nanopore sensors.

The present technology can be further summarized by the following list of features.

1. A device for sequencing biopolymers, the device comprising,

a first MXene layer configured as an electrode;

a second MXene layer disposed on a surface of the first MXene layer;

an interlayer space between the first and second MXene layers;

an insulator layer disposed on a surface of the second MXene layer opposite the interlayer space;

a first electrolyte solution chamber configured to contain electrolyte solution in contact with a surface of the first MXene layer opposite the interlayer space;

a solution electrode disposed in the first electrolyte solution chamber.

a second electrolyte solution chamber configured to contain electrolyte solution in contact with said insulator layer; and

a nanopore penetrating through the first MXene layer, the interlayer space, the second MXene layer, and the insulator layer, and forming a conductive pathway between the first and second electrolyte chambers.

2. The device of feature 1, wherein the first and second MXene layers each comprise an MXene material independently selected from the group consisting of Ti₂C, V₂C, Cr₂C, Nb₂C, Ta₂C, Ti₃C₂, V₃O₂, Ta₃C₂, Ti₄C₃, V₄O₃, Nb₄C₃, Ta₄C₃, Mo₂TiC₂, Cr₂TiC₂, and Mo₂Ti₂C₃.
3. The device of feature 1 or feature 2, wherein the first and second MXene layers each has a thickness in the range from one to about five atoms and a surface area in the range from about 0.001 to about 10,000 mm².
4. The device of any of the preceding features, wherein the insulator layer comprises a material selected from the group consisting of Al₂O₃, TiO₂, HfO₂, VO₂, SiO₂, and BN and has a thickness in the range from about 0.5 to about 5 nm.
5. The device of any of the preceding features, wherein the nanopore has a diameter in the range from about 0.3 nm to about 10 nm.
6. The device of any of the preceding features, wherein the first MXene layer is in electrical contact with a conductive metal contact configured for electrical connection to a voltage source.
7. The device of any of the preceding features, wherein the first and/or second electrolyte chamber comprises silicon nitride.
8. The device of any of the preceding features, wherein the interlayer space comprises a plurality of cations.
9, The device of any of the preceding features, further comprising a solution electrode disposed in the second electrolyte chamber.
10. A device for sequencing biopolymers, the device comprising,

a first MXene layer configured as an electrode and contacting a first electrical contact layer;

a second MXene layer disposed on a surface of the first MXene layer opposite the first electrical contact layer;

a first interlayer space between the first and second MXene layers;

a first insulator layer disposed on a surface of the second MXene layer opposite the interlayer space;

a third MXene layer disposed on a surface of the first insulator layer opposite the second MXene layer;

a fourth MXene layer disposed on a surface of the third MXene layer opposite the first insulator layer;

a second interlayer space between the third and fourth MXene layers;

an electrical contact layer disposed on a surface of the fourth MXene layer opposite the second interlayer space;

a second insulator layer disposed on a surface of the electrical contact layer opposite the fourth MXene layer;

a first electrolyte solution chamber configured to contain electrolyte solution in contact with a surface of the first MXene layer opposite the first interlayer space;

a second electrolyte solution chamber configured to contain electrolyte solution in contact with the second insulator layer; and

a nanopore penetrating through the first electrical contact layer, the first MXene layer, the first interlayer space, the second MXene layer, the first insulator layer, the third MXene layer, the second interlayer space, the fourth MXene layer, the second electrical contact layer, and the second insulator layer, and forming a conductive pathway between the first and second electrolyte chambers.

11. The device of feature 10, wherein the first, second, third, and fourth MXene layers each comprise an MXene material independently selected from the group consisting of Ti₂C, V₂C, Cr₂C, Nb₂C, Ta₂C, Ti₃C₂, V₃O₂, Ta₃C₂, Ti₄C₃, V₄O₃, Nb₄C₃, Ta₄C₃, Mo₂TiC₂, Cr₂TiC₂, and Mo₂Ti₂C₃.
12. The device of feature 10 or feature 11, wherein the first, second, third, and fourth MXene layers each has a thickness in the range from one to about five atoms and a surface area in the range from about 0.001 to about 10,000 mm².
13. The device of any of features 10-12, wherein the first and second insulator layers each comprises a material independently selected from the group consisting of Al₂O₃, TiO₂, HfO₂, VO₂, SiO₂, and BN and has a thickness in the range from about 0.5 to about 5 nm.
14. The device of any of features 10-13, wherein the nanopore has a diameter in the range from about 0.3 nm to about 10 nm.
15. The device of any of features 10-14, wherein the first and/or second electrolyte chamber comprises silicon nitride.
16. The device of any of features, wherein the first and/or second interlayer space comprises a plurality of cations.
17, The device of any of features 10-16, further comprising a solution electrode disposed in the first electrolyte chamber and a solution electrode disposed in the second electrolyte chamber.
18. A method of sequencing a biopolymer, the method comprising,

(a) providing the device of any of the preceding features, a voltage source, an amplifier, an electrolyte solution, and a biopolymer;

(b) optionally processing the biopolymer by a method that comprises denaturation and/or fragmentation;

(c) depositing the electrolyte solution into the first and second electrolyte solution chambers of the device and depositing the biopolymer or processed biopolymer into the electrolyte solution in the first electrolyte solution chamber;

(d) applying a voltage difference between the first and second electrolyte solution chambers, thereby causing a single molecule of the biopolymer to move through the nanopore of the device and causing current flow through the nanopore;

(e) measuring a change in current flow associated with the passage of monomer units of the biopolymer through the nanopore; and

(f) correlating the change in current flow with a known change in current flow characteristic of passage of a specific type of monomeric unit through the nanopore, thereby determining the identity of the monomer;

(g) repeating steps (e) and (f) to determine a sequence of monomeric units of the biopolymer.

19. The method of feature 18, wherein the biopolymer is a DNA, RNA, protein, or peptide.
20. The method of feature 18 or 19, further comprising:

(c1) applying a negative voltage to an MXene electrode of the device, thereby causing cations from the electrolyte solution to move into an interlayer of the device and charging the MXene electrode with a plurality of cations.

21. The method of feature 20, whereby the charged MXene electrode supplies cations for current flow through the nanopore during steps (d) and (e).
22. The method of any of features 18-21, wherein the device comprises a solution electrode in each electrolyte solution chamber, and the voltage applied in step (d) is applied between the solution electrodes, while ionic current through the nanopore is driven by a separate voltage applied between an MXene electrode and a solution electrode, or between two MXene electrodes.
23. The method of any of features 18-22, wherein no access resistance impedes ionic current flow through the nanopore during steps (d) and (e).
24. The method of any of features 18-23, wherein the voltage applied in steps (d) and (e) to drive ionic current through the nanopore is a DC voltage, an AC voltage, or a combination of DC and AC voltages.
25. The method of any of features 18-24, wherein cations stored in an interlayer space become depleted, and the method comprises applying a negative potential to an MXene electrode to recharge the interlayer space with cations.

### BRIEF DESCRIPTION OF DRAWINGS

FIG. 1A shows a schematic representation of a prior art graphene nanopore device for use in sequencing a single-stranded nucleic acid molecule. Influence of access resistance (Ra) on the total pore resistance (Rp) zone limits resolution of conductance changes caused by structures within the pore. Ions moving from each bulk chamber to the other face access resistance (total resistance=Rp+2Ra). Arrows show the direction of cation and anion movement upon applying voltage. FIG. 1B shows a schematic representation of an MXene nanopore device of the present technology, in which one of the two MXene membranes serves as one of the two working electrodes. Ions only moving from the bulk chamber to the MXene interlayer space face access resistance (total resistance=Rp+Ra). Arrows show the direction of cation movement upon applying positive voltage to the MXene electrode. FIG. 1C shows a schematic illustration of an MXene nanopore device in which the outer two MXene membranes are used as the two working electrodes. Ions moving in either direction, i.e., from one interlayer space to the other, face only the pore resistance and no access resistance (total resistance=Rp). Arrows show direction of cation movement in either direction. For example, cations can flow from the upper interlayer space to the lower interlayer space upon applying positive voltage to the upper electrode and negative voltage to the lower electrode, and vice versa. Alternating current (AC) mode can also be used, in which the voltage between the top and bottom electrodes is oscillated with time, producing a flow of ions across the electrodes.

FIG. 2 shows a schematic diagram of a single-stranded DNA molecule threaded and driven base-by-base through an MXene nanopore using an enzyme. Examples of such enzymes include DNA helicases and DNA polymerases which can ratchet along a DNA molecule in single-base increments. The enzyme is not attached chemically to the electrode, but is held there because of the applied force on the DNA molecule from the trans-nanopore voltage.

FIG. 3 shows a schematic diagram of a protein molecule being unfolded and passed through an MXene nanopore using an enzyme. Enzymes such as any one of the class of unfoldase proteins (e.g., CIpX) can be used to hold the protein in the pore.

FIG. 4 shows a schematic illustration of an MXene nanopore immersed in a solution of water and salt.

FIG. 5 shows the current measured through a nanopore in an MXene device during changes in the voltage applied between the working electrodes. The upper trace shows a decrease of current (reflecting a decrease of conductance) upon applying high voltage; the decrease was due to extraction of cations from the MXene interlayer space. Partial recovery was seen after return to lower voltage. The lower trace shows the change of MXene membrane thickness measured from the change in conductance.

FIG. 6A shows an AFM image of a transferred MXene flake on an atomically-flat highly-oriented pyrolytic graphite (HOPG) surface. The white circles in the image represent trapped aqueous solution underneath the flake. FIG. 6B shows a change of flake height as a function of voltage, which indicates ion intercalation and de-intercalation. A Keithley voltage source was used to apply voltage between the HOPG support and a Ag/AgCl electrode immersed in the same electrolyte solution (0.4 M KCl solution).

FIG. 7A is a schematic illustration of wafer-scale transfer of self-assembled MXene flakes onto a substrate. FIG. 7B is an AFM image of a self-assembled monolayer of MXene flakes. which shows the tiling of monolayer flakes into a mosaic with gaps between flakes, forming an area with >90% monolayer coverage. FIG. 7C shows an SEM image of the same self-assembled monolayer of MXene flakes as in FIG. 7B. Contrast in the image corresponds to either a different orientation or adhesion of the MXene flakes to the substrate.

FIG. 8 shows the measured sheet resistances of monolayer, bilayer, and trilayer Ti₃C₂ films (two different samples, 1 and 2, were measured) using a four-probe Van der Pauw measurement method performed four times on each sample. See van der Pauw, L. J., Philips Research Reports. 13: 1-9 (1958). In this measurement, four electrodes with square geometry in 1 cm×1 cm area were placed on the film and resistance was measured along each line of the square (two vertical lines and two horizontal lines). The conductivity of the single-layer MXene film, whose thickness was verified using AFM measurements, confirms that electrons are delivered through macroscale electrodes to the MXene sheets at the pore in the devices of the present technology.

### DETAILED DESCRIPTION

The present technology provides a nanopore electrode sequencer for the characterization and sequencing of biomolecules. The technology utilizes two or more MXene sheets or membranes containing nanopores. The devices offer low cost biodiagnostics and sequencing with high resolution, high accuracy, rapid single molecule sequencing, and high throughput. The devices offer higher resolution than previous single molecule nanopore-based sequencing technologies due to reduction or elimination of access resistance to ions entering the nanopore from bulk solution. Instead, ions for transit through the pore are provided from cations accumulated in an interlayer space between MXene sheets. Using a suitable configuration of MXene sheets, electrodes, nanopores, and insulation layers, access resistance can be substantially reduced or eliminated with the present technology.

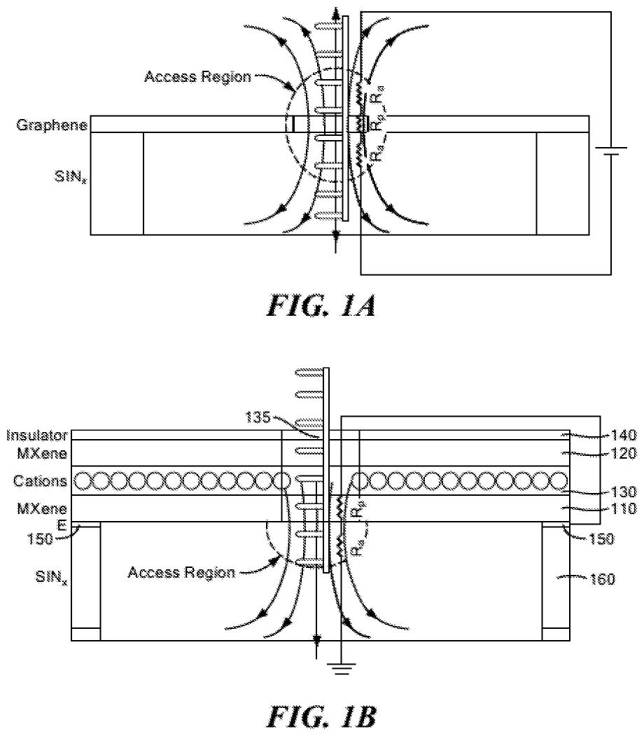

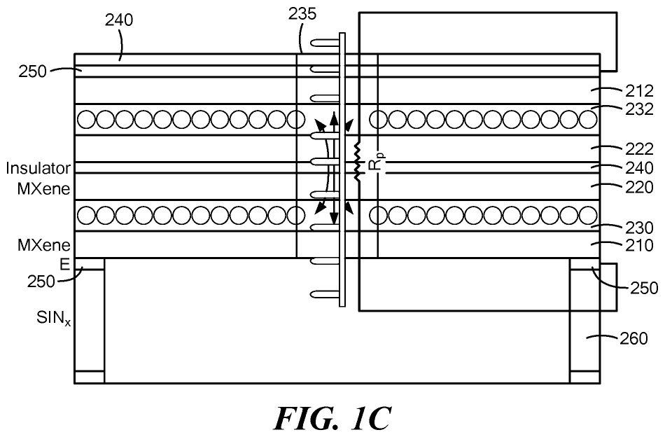

In a first configuration, a solid-state 2D MXene material, such as a material comprising or consisting of Ti₂C, V₂C, Cr₂C, Nb₂C, Ta₂C, Ti₃C₂, V₃C₂, Ta₃C₂, Ti₄C₃, V₄C₃, Nb₄C₃, Ta₄C₃, Mo₂TiC₂, Cr₂TiC₂, or Mo₂Ti₂C₃, (MXene-electrode layer 110) is used as one of the working electrodes to which a potential is directly applied. Another 2D MXene layer (MXene-insulator layer 120) is superimposed over the MXene-electrode layer, leaving interlayer space 130 between the MXene-electrode layer and the MXene-insulator layer. Each of the MXene layers can be from 1-5 atoms thick, and is preferably 1-2 atoms thick or 1 atom thick. The surface area of the MXene layers can be selected according to need, and can be, for example, about 0.001 to about 10,000 mm². Insulator layer 140, containing or consisting of an electrically insulating material such as Al₂O₃, TiO₂, HfO₂, VO₂, SiO₂, BN, e.g. a metal oxide or nitride, or other thin insulating layer having a thickness in the range from 0.5 to 5 nm, is deposited onto the surface of the MXene-insulator layer opposite the interlayer space. See FIG. 1B. Nanopore 135 traverses both MXene layers and the insulating layer. The nanopore can have a diameter in the range from about 0.3 nm to about 10 nm. The nanopore can be introduced using an electron beam, an ion beam, a laser, or another method. A solution electrode is immersed in an electrolyte buffer (e.g., an aqueous solution containing KCl, NaCl, LiCl, CaCl₂, MgCl₂, or another salt, either alone or combined) for the application of voltage between the MXene-electrode layer and the solution electrode. The MXene-electrode layer is in contact with conductive material 150, such as a conductive metal (e.g., Au, Ag, Cu, Cr, or mixtures thereof) or a conductive polymer, to provide electrical continuity with a device such as an amplifier for setting constant voltage conditions and measuring current between the electrodes. The electrolyte buffer can be contained in chamber or well 160, which can be formed of a non-conductive material, such as silicon nitride.

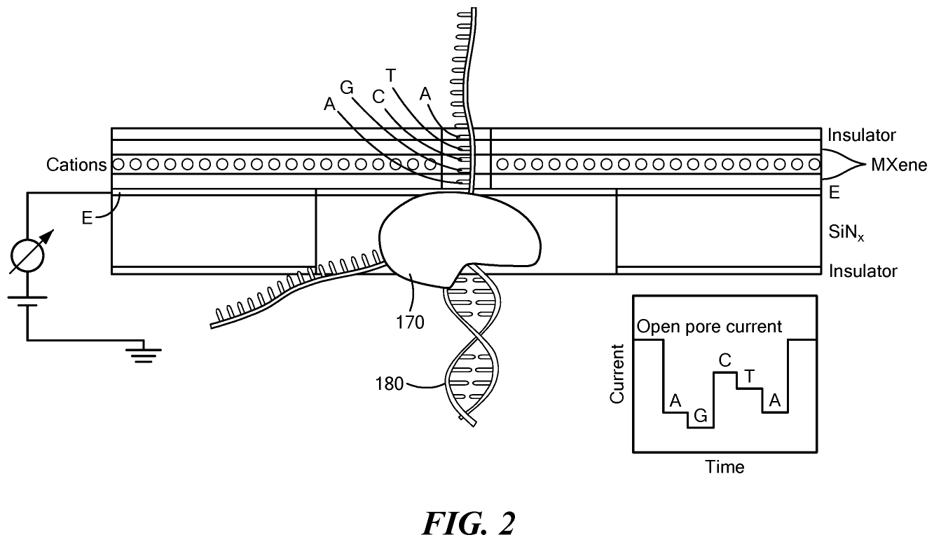

In the first configuration, by applying negative voltage to the MXene-electrode layer (also referred to herein as the “MXene electrode”) and positive voltage to the solution electrode, cations move from solution toward the MXene electrode and intercalate between the layers. This is the charging state. When, the voltage is reversed, cations move from the interlayer space toward the solution, creating steady ionic current through the nanopore. For a biopolymer sequencing process, DNA, RNA, or a protein molecule bound to enzyme 170 that ratchets biopolymer 180 base by base or amino acid by amino acid (for example, a helicase or a DNA or RNA polymerase, or an unfoldase) is added to the electrolyte chamber and is pulled toward the pore electrokinetically (either by electrophoresis or electroosmosis, or both). See FIG. 2. Then, the ratcheting enzyme unwinds and threads monomeric units one at a time through the pore. This causes a reduction in the number of ions passing between the electrodes, leading to reduction in the current detected by an amplifier. The amount of the current reduction is proportional to the size of the bases (for example, A, C, T, G for DNA, and A, U, C, G for RNA), or other monomeric units, which helps distinguish the bases or monomeric units, allowing sequencing of the biopolymer. This design eliminates the problem of access resistance encountered when ions from solution enter into atomically thin pores, which considerably reduces sensing resolution. If the interlayer space becomes discharged during a measurement, then it can be recharged during a measurement or between measurements by briefly reversing the voltage polarity to restore the charged state, followed by returning to the voltage polarity used for measurement of ionic current through the nanopore.

In a second configuration, a solid-state 2D MXene material, such as a material comprising or consisting of Ti₂C, V₂C, Cr₂C, Nb₂C, Ta₂C, Ti₃C₂, V₃C₂, Ta₃C₂, Ti₄C₃, V₄C₃, Nb₄C₃, Ta₄C₃, Mo₂TiC₂, Cr₂TiC₂, or Mo₂Ti₂C₃, (first MXene-electrode layer 210, or first MXene electrode) is used as one of the working electrodes to which a potential is directly applied. See FIG. 1C. Another 2D MXene layer (first MXene-insulator layer 220) is superimposed over the first MXene-electrode layer, leaving first interlayer space 220 between the first MXene-electrode layer and the first MXene-insulator layer. Insulator layer 240, containing or consisting of an electrically insulating material such as Al₂O₃, TiO₂, HfO₂, VO₂, SiO₂, BN, or other insulating thin layer, is deposited over the first MXene insulator layer. Then, a second pair of MXene layers are deposited over the insulating layer on a surface of the insulating layer opposite the first MXene insulator layer. The second pair of MXene layers include second MXene insulator layer 222 and second MXene electrode layer 212, which are separated by second interlayer space 232. Another Insulator layer 240, containing or consisting of an electrically insulating material such as Al₂O₃, TiO₂, HfO₂, VO₂, SiO₂, BN, e.g. a metal oxide or nitride, or other thin insulating layer having a thickness in the range from 0.5 to 5 nm, is deposited onto the surface of the second MXene-insulator layer opposite the second interlayer space. Nanopore 235 traverses all four MXene layers, the two insulating layers, and both conductive contacts. The nanopore can have a diameter in the range from about 0.3 nm to about 10 nm. The nanopore can be introduced using an electron beam, an ion beam, a laser, or another method. The first and second MXene electrodes are connected via metal contacts 250 to opposite sides of a voltage source; there is no solution electrode required in this configuration to measure ionic currents through the nanopore, once at least one of the interlayers has been charged with cations. The electrolyte buffer can be contained in chamber or well 260, which can be formed of a non-conductive material, such as silicon nitride. See FIG. 10.

In the second configuration, one electrode is charged by applying negative voltage to the electrode and positive voltage to an electrolyte solution exposed to the nanopore. As a result, cations move through the nanopore, toward the negative electrode, and intercalate in the interlayer space adjacent to the negative electrode (charging state). To then measure ionic current through the nanopore, positive voltage is applied to the charged MXene electrode and negative voltage to the other MXene electrode, prompting cations to move from charged electrode to the uncharged electrode, creating steady ionic current. As for the first configuration, a biopolymer 280 such as DNA, RNA, or a protein molecule bound to an enzyme 270 (a helicase or DNA or RNA polymerase, or an unfoldase) that ratchets the biopolymer base by base can be added to the electrolyte chamber and is pulled toward the pore. Then, the ratcheting enzyme unwinds and threads DNA or RNA bases or protein amino acids one at a time through the pore, leading to reduction in the current. The amount of the current reduction is proportional to the size of the monomeric units, allowing sequencing of the biopolymer. This design also eliminates the problem of access resistance encountered when ions from solution enter into nanopores.

Methods for producing thin layers of MXene material are known, and any such method can be used to produce the MXene films used in the present technology. See, e.g., Naguib, M., et al., Advanced Materials 23 (37):4248-4253 (2011). MXenes are transition metal carbides or nitrides, or carbonitrides, and are generally both hydrophilic and electrically conductive. MXenes can be produced by selectively etching out the A element, e.g., using HF, from a material having the general formula Mₙ₊₁AXₙ, where M is an early transition metal, A is an element from group 13 or 14 of the periodic table, X is C and/or N, and n=1-4. See, e.g., Deysher, G., et al., ACS Nano 14 (1):204-217 (2019). MXenes also can be produced using mixtures of two different transition metals. MXene material can be delaminated to produce single layer flakes using ultrasound treatment or treatment with DMSO and stirring. See Mashtalir, O., et al., Nature Communications. 4:1716 (2013).

FIG. 1A shows the access region around a conventional nanopore, which gives rise to access resistance which forms a component of the total resistance through the pore. In a graphene nanopore as shown in FIG. 1A, upon applying voltage, ions moving from each bulk chamber to the other encounter access resistance. Therefore, the total resistance through the pore is the sum of the pore resistance (Rp) and both of the access resistances (2*Ra). In the MXene nanopore device shown in FIG. 1B, ions encounter access resistance only in moving from the bulk chamber toward the MXene interlayer space, i.e., during charging of the MXene interlayer space. Ions do not face any access resistance by moving from the MXene interlayer space to the bulk chamber. Therefore, the total resistance through the MXene pore of the present technology is less than in the case of a graphene nanopore, which includes access resistance (2Ra). In the MXene nanopore device shown in FIG. 1C, ions moving from one MXene interlayer space toward other interlayer space do not encounter any access resistance. Therefore, the voltage drop across the pore is the largest in the case of the MXene pores in this configuration.

FIG. 2 schematically shows a single-stranded DNA being threaded and driven base-by-base through a nanopore using an enzyme. In this design, intercalating 2D materials are used as one of the working electrodes, and ion transport from within the MXene interlayer space to the bottom bulk chamber provides the ionic current signal. The model current trace shows a base-by-base DNA sequencing event wherein the sequence of bases is identified by the unique current blockage for each base.

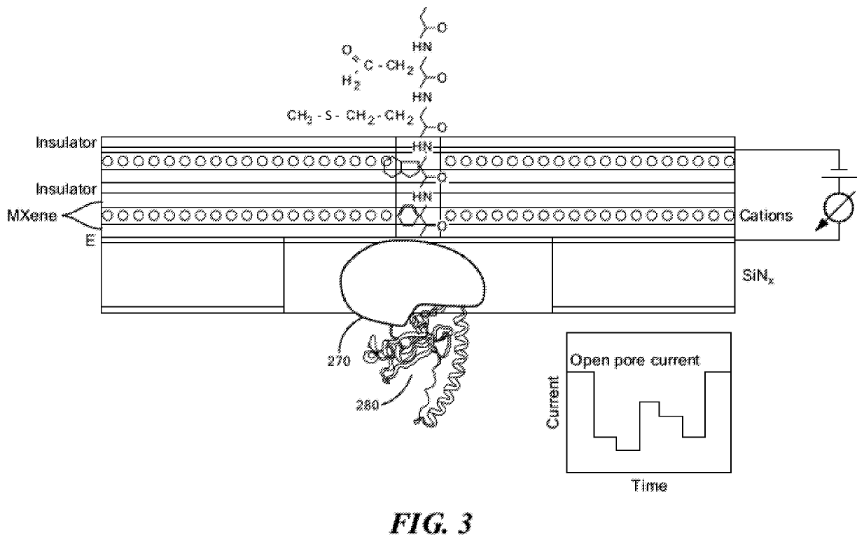

FIG. 3 schematically shows a protein molecule being unfolded and passed through an MXene nanopore using a protein-processing enzyme (e.g., an unfoldase). In this design, intercalating 2D materials are used as working electrodes. Ion transport from within one of the MXene electrodes to the to the other MXene electrode provides the signal. The model current trace shows amino acid sequencing of the protein molecule based on the current blockage obtained for each amino acid.

An optional feature for use with any of the devices described above is the inclusion of a pair of solution electrodes, a first solution electrode present in the lower electrolyte chamber and a second solution electrode present in the upper electrolyte chamber. This pair of electrodes can be used to provide a driving voltage for elongating and stretching the biopolymer to aid its entry into the nanopore or for threading and displacement of the biopolymer once in the nanopore. The advantage of using this additional pair of electrodes is that an electric field can be established over a larger space than if only the electrodes at the MXene films were used. The additional pair of electrodes can be any conventional electrodes for use in establishing a voltage and current flow through an electrolyte solution; for example, Ag/AgCl electrodes can be used. The additional pair of electrodes preferably are driven by a separate voltage source from that used to set the voltage and measure current between the MXene electrode and its solution electrode, or between first and second MXene electrodes.

The devices and methods described herein have several advantageous features compared to previous nanopore-based biopolymer sequencing technologies. The MXene nanopore technology uses a nanometer-thick free-standing membrane, assembled from two-dimensional materials. The use of synthetic materials instead of polymer-embedded proteins results in higher mechanical stability, durability, and robustness. Further, unlike most 2D materials, MXenes are hydrophilic, which is more biocompatible for biomolecule analysis than most 2D materials. The MXene flakes can be conveniently self-assembled to form a freestanding two-dimensional material using a simple solvent-solvent interface method. Moreover, due to their electrical conductivity and cation binding capacity, MXene films can be used as electrodes that bind and release cations. Layered MXene films can Intercalate cations in their interlayer spaces, and the cations can be released by applying reverse voltage to obtain a steady local ionic current through the pore, thereby eliminating access resistance at the mouth of the nanopore and maximizing resolution of ionic currents through the nanopore. By maximizing resolution of changes in pore current, more detailed information can be obtained, enabling improved or more complex biopolymer sequencing and other analyses not previously practical or reliable. The thickness of a nanopore-containing MXene membrane can be dynamically changed based on the applied voltage across the membrane. MXene electrodes contract upon intercalation of cations, leading to lower thickness, and expand upon releasing cations, leading to higher thickness; this property may be used to control the resolution of the readout, or to facilitate rapid loading of ions into the MXene interstitial region for further sequencing.

The present technology can be used to perform long-read sequencing of single DNA, RNA, or protein molecules with either multi-base or single-base resolution. The elimination of access resistance at the nanopore makes possible the detection of a greater set of modifications in RNA and proteins than possible using previous nanopore technology. Structural analysis of DNA, RNA, proteins, and other biomolecules is also possible, and long-read mapping of DNA sequences by sequence-specific tagging can be performed. Parallelization of multiple MXene nanopore devices will lead to increased yield, reduced cost, and improved accuracy of sequencing due to multiplexed analysis of the same molecule in several devices simultaneously.

### EXAMPLES

### Example 1. Cation Flow from MXene Interlayer Space

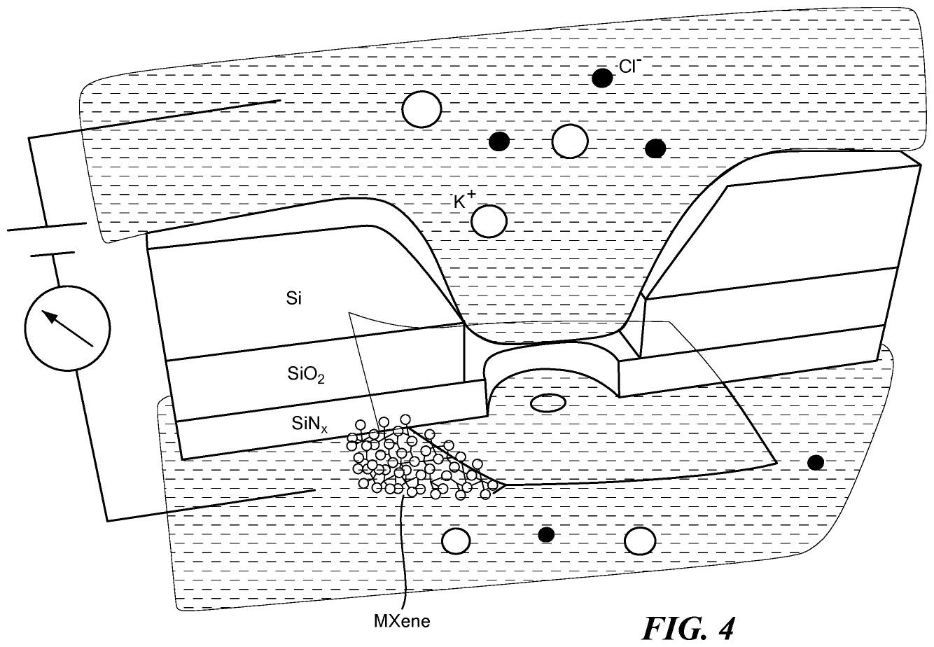

A conventional nanopore set-up was fitted with a freestanding MXene bilayer membrane through which a nanopore had been drilled with an electron beam (FIG. 4). K⁺ ions from an aqueous KCl solution were intercalated into the interlayer space between two Ti₃C₂ flakes, and also were removed from the interlayer space, as shown below.

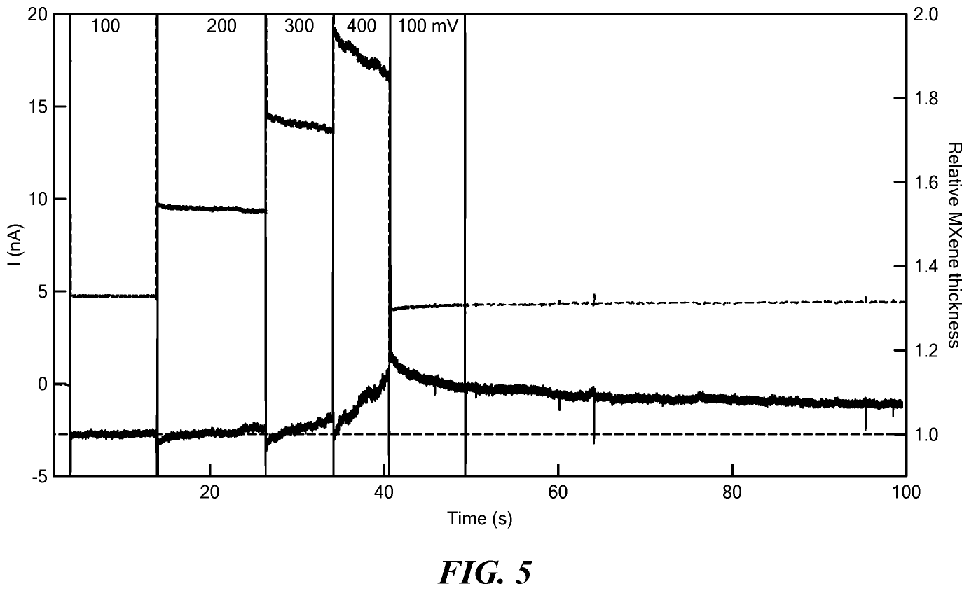

FIG. 5 shows an experiment performed with two adjacent Ti₃C₂ MXene membranes having a combined nanopore that was 6 nm in diameter and 3 nm thick. The upper trace presents current as a function of time, and the lower trace shows how the relative nanopore thickness changed over time, measured purely from the change in conductance. According to the 100 mV data in the first 10 seconds, the conductance was 47 nS at 0.4 M KCl. Then, by doubling voltage to 200 mV, the conductance initially doubled but then decreased, which is believed to be due to the expulsion of K⁺ ions from the MXene interlayer space. The same phenomenon was observed at higher voltages, except that the rate of cation expulsion increased. Finally, by going back to 100 mV, partial recovery in the conductance was observed, which indicates repopulation of the MXene interlayer space with cations. The reduction in conductance corresponds to a 20% increase in MXene membrane thickness when ions were expelled from within the sheet, (approximately 0.6 nm increase in the 3 nm initial film thickness). The increase in conductance after lowering the voltage to 100 mV at the end of the trace corresponds to a 50% recovery in thickness.

### Example 2. In Situ Measurement of MXene Membrane Thickness by AFM

Intercalation of cations between MXene flakes was shown as change of MXene membrane thickness using in-situ atomic force microscopy (AFM) while applying voltage across the juxtaposed MXene membranes.

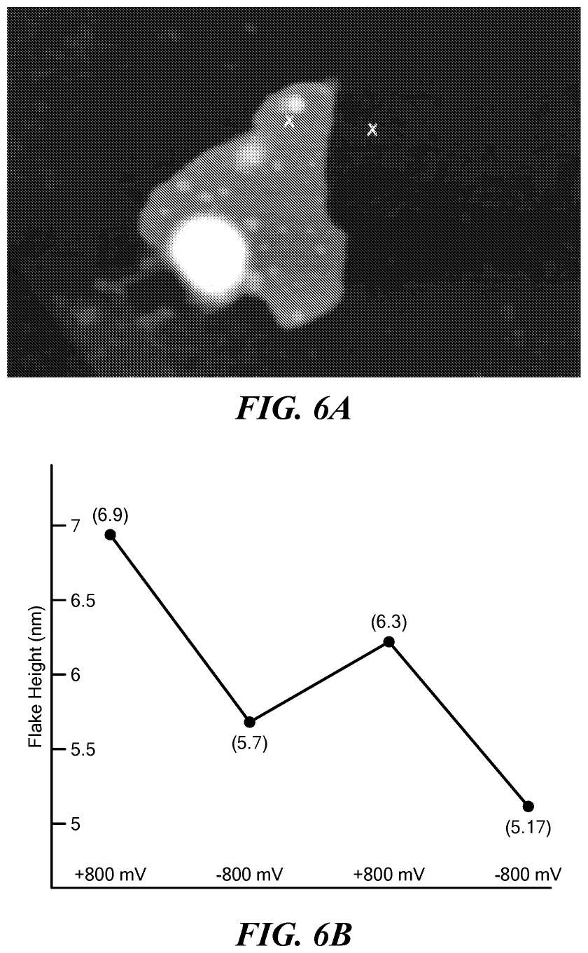

MXene flakes were transferred onto a highly oriented pyrolytic graphite (HOPG) surface to form a few-layer thick multi-flake assembly. One electrode was connected to the HOPG and the other electrode was immersed in a buffer droplet (0.4M KCl) placed on the HOPG surface and covering the MXene flake assembly. Voltage was reversed several times and its effect on thickness of the membrane was measured. The results showed that applying negative voltage to the film causes the cations to intercalate between MXene layers leading to shrinking of pore thickness. By reversing voltage, cations were expelled from the layers resulting in expansion of membrane thickness, as shown in FIG. 6B, in which the thickness of the assembly was monitored using AFM.

### Example 3. Assembly of Wafer Scale Freestanding MXene Membranes Using Solvent-Solvent Interface Method

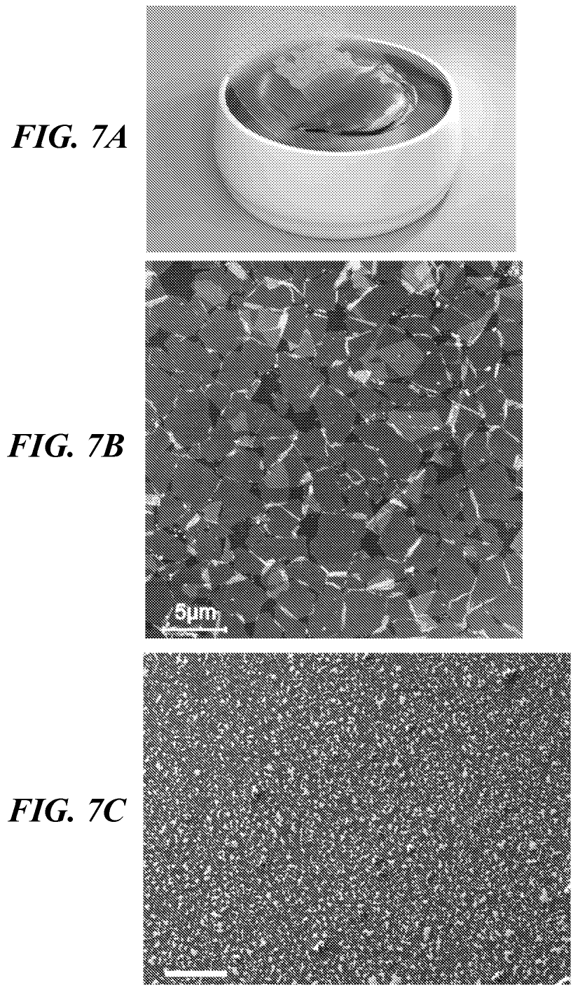

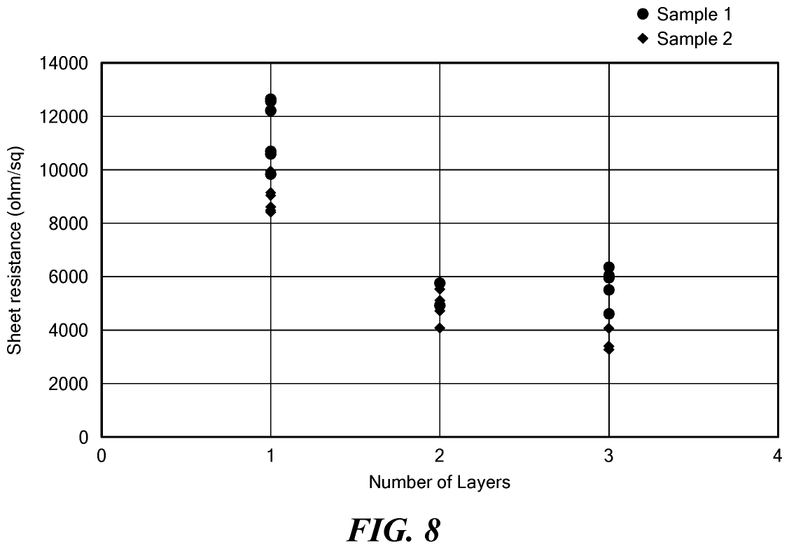

Monolayer MXene flakes of Ti₃C₂ were self-assembled at a chloroform/methanol/water interface. First, an MXene dispersion was prepared in a methanol:water (8:1) mixture (final concentration of methanol was about 12% by volume). This dispersion was layered onto chloroform, allowing the formation of an interfacial MXene film. After assembly, the film could be transferred onto a substrate of choice, such as a silicon wafer, by either lifting the substrate up through the liquid-liquid interface (e.g., from the chloroform phase upwards through the interface), or by lowering the chloroform interface through removal of chloroform from the bottom phase. When this is performed properly, the arrangement of flakes in the film is not disturbed. FIG. 7A shows a schematic illustration of a wafer-scale transfer process. FIGS. 7B and 7C show an AFM image and an SEM image of the Ti₃C₂ film respectively.

As used herein, “consisting essentially of” allows the inclusion of materials or steps that do not materially affect the basic and novel characteristics of the claim. Any recitation herein of the term “comprising”, particularly in a description of components of a composition or in a description of elements of a device, can be exchanged with the alternative expressions “consisting essentially of” or “consisting of”.

What is claimed is:

1. A device for sequencing biopolymers, the device comprising,
a first MXene layer configured as an electrode;
a second MXene layer disposed on a surface of the first MXene layer;
an interlayer space between the first and second MXene layers;
an insulator layer disposed on a surface of the second MXene layer opposite the interlayer space;
a first electrolyte solution chamber configured to contain electrolyte solution in contact with a surface of the first MXene layer opposite the interlayer space;
a solution electrode disposed in the first electrolyte solution chamber.
a second electrolyte solution chamber configured to contain electrolyte solution in contact with said insulator layer; and
a nanopore penetrating through the first MXene layer, the interlayer space, the second MXene layer, and the insulator layer, and forming a conductive pathway between the first and second electrolyte chambers.

2. The device of claim 1, wherein the first and second MXene layers each comprise an MXene material independently selected from the group consisting of Ti₂C, V₂C, Cr₂C, Nb₂C, Ta₂C, Ti₃C₂, V₃C₂, Ta₃C₂, Ti₄C₃, V₄C₃, Nb₄C₃, Ta₄C₃, Mo₂TiC₂, Cr₂TiC₂, and Mo₂Ti₂C₃.

3. The device of claim 1, wherein the first and second MXene layers each has a thickness in the range from one to about five atoms and a surface area in the range from about 0.001 to about 10,000 mm².

4. The device of claim 1, wherein the insulator layer comprises a material selected from the group consisting of Al₂O₃, TiO₂, HfO₂, VO₂, SiO₂, and BN and has a thickness in the range from about 0.5 to about 5 nm.

5. The device of claim 1, wherein the nanopore has a diameter in the range from about 0.3 nm to about 10 nm.

6. The device of claim 1, wherein the first MXene layer is in electrical contact with a conductive metal contact configured for electrical connection to a voltage source.

7. The device of claim 1, wherein the first and/or second electrolyte chamber comprises silicon nitride.

8. The device of claim 1, wherein the interlayer space comprises a plurality of cations.

9. The device of claim 1, further comprising a solution electrode disposed in the second electrolyte chamber.

10. A device for sequencing biopolymers, the device comprising,
a first MXene layer configured as an electrode and contacting a first electrical contact layer;
a second MXene layer disposed on a surface of the first MXene layer opposite the first electrical contact layer;
a first interlayer space between the first and second MXene layers;
a first insulator layer disposed on a surface of the second MXene layer opposite the interlayer space;
a third MXene layer disposed on a surface of the first insulator layer opposite the second MXene layer;
a fourth MXene layer disposed on a surface of the third MXene layer opposite the first insulator layer;
a second interlayer space between the third and fourth MXene layers;
an electrical contact layer disposed on a surface of the fourth MXene layer opposite the second interlayer space;
a second insulator layer disposed on a surface of the electrical contact layer opposite the fourth MXene layer;
a first electrolyte solution chamber configured to contain electrolyte solution in contact with a surface of the first MXene layer opposite the first interlayer space;
a second electrolyte solution chamber configured to contain electrolyte solution in contact with the second insulator layer; and
a nanopore penetrating through the first electrical contact layer, the first MXene layer, the first interlayer space, the second MXene layer, the first insulator layer, the third MXene layer, the second interlayer space, the fourth MXene layer, the second electrical contact layer, and the second insulator layer, and forming a conductive pathway between the first and second electrolyte chambers.

11. The device of claim 10, wherein the first, second, third, and fourth MXene layers each comprise an MXene material independently selected from the group consisting of Ti₂C, V₂C, Cr₂C, Nb₂C, Ta₂C, Ti₃C₂, V₃C₂, Ta₃C₂, Ti₄C₃, V₄O₃, Nb₄C₃, Ta₄C₃, Mo₂TiC₂, Cr₂TiC₂, and Mo₂Ti₂C₃.

12. The device of claim 10, wherein the first, second, third, and fourth MXene layers each has a thickness in the range from one to about five atoms and a surface area in the range from about 0.001 to about 10,000 mm².

13. The device of claim 10, wherein the first and second insulator layers each comprises a material independently selected from the group consisting of Al₂O₃, TiO₂, HfO₂, VO₂, SiO₂, and BN and has a thickness in the range from about 0.5 to about 5 nm.

14. The device of claim 10, wherein the nanopore has a diameter in the range from about 0.3 nm to about 10 nm.

15. The device of claim 10, wherein the first and/or second electrolyte chamber comprises silicon nitride.

16. The device of claim 10, wherein the first and/or second interlayer space comprises a plurality of cations.

17. The device of claim 10, further comprising a solution electrode disposed in the first electrolyte chamber and a solution electrode disposed in the second electrolyte chamber.

18. A method of sequencing a biopolymer, the method comprising,
(a) providing the device of any of the preceding claims, a voltage source, an amplifier, an electrolyte solution, and a biopolymer;
(b) optionally processing the biopolymer by a method that comprises denaturation and/or fragmentation;
(c) depositing the electrolyte solution into the first and second electrolyte solution chambers of the device and depositing the biopolymer or processed biopolymer into the electrolyte solution in the first electrolyte solution chamber;
(d) applying a voltage difference between the first and second electrolyte solution chambers, thereby causing a single molecule of the biopolymer to move through the nanopore of the device and causing current flow through the nanopore;
(e) measuring a change in current flow associated with the passage of monomer units of the biopolymer through the nanopore; and
(f) correlating the change in current flow with a known change in current flow characteristic of passage of a specific type of monomeric unit through the nanopore, thereby determining the identity of the monomer;
(g) repeating steps (e) and (f) to determine a sequence of monomeric units of the biopolymer.

19. The method of claim 18, wherein the biopolymer is a DNA, RNA, protein, or peptide.

20. The method of claim 18, further comprising:
(c1) applying a negative voltage to an MXene electrode of the device, thereby causing cations from the electrolyte solution to move into an interlayer of the device and charging the MXene electrode with a plurality of cations.

21. The method of claim 20, whereby the charged MXene electrode supplies cations for current flow through the nanopore during steps (d) and (e).

22. The method of claim 18, wherein the device comprises a solution electrode in each electrolyte solution chamber, and the voltage applied in step (d) is applied between the solution electrodes, while ionic current through the nanopore is driven by a separate voltage applied between an MXene electrode and a solution electrode, or between two MXene electrodes.

23. The method of claim 18, wherein no access resistance impedes ionic current flow through the nanopore during steps (d) and (e).

24. The method of claim 18, wherein the voltage applied in steps (d) and (e) to drive ionic current through the nanopore is a DC voltage, an AC voltage, or a combination of DC and AC voltages.

25. The method of any of claim 18, wherein cations stored in an interlayer space become depleted, and the method comprises applying a negative potential to an MXene electrode to recharge the interlayer space with cations.
